(19) United States
(12) Patent Application Publication
(10) Pub. No.: US 2010/0012832 A1
(43) Pub. Date: Jan. 21, 2010

(54) METHOD OF SEPARATING PHOSPHORYLATED PEPTIDE OR PHOSPHORYLATED PROTEIN

(75) Inventor: Yasushi Ishihama, Yamagata (JP)

Correspondence Address: FOLEY AND LARDNER LLP;SUITE 500, 3000 K STREET NW, WASHINGTON, DC, 20007, US

(73) Assignee: Keio University

(21) Appl. No.: 12/374,966

(22) PCT Filed: Aug. 9, 2007
(86) PCT No.: PCT/JP2007/065923

§ 371 Date: Jul. 7, 2009

(30) Foreign Application Priority Data
Aug. 17, 2006 (JP) 2006-222316

(51) Int. Cl. B01D 59/44; C07K 1/16; C01G 23/04

(52) U.S. Cl. 250/282; 530/415; 530/344; 423/609

(57) ABSTRACT

According to the present invention, phosphorylated peptides and/or phosphorylated proteins are specifically separated. A sample containing a phosphorylated peptide and/or a phosphorylated protein is supplied to a separation unit filled with a metal oxide in the presence of an aliphatic hydroxycarboxylic acid. Upon separation of a phosphorylated peptide and/or a phosphorylated peptide with the use of a separation unit filled with a metal oxide, adsorption of carboxylic acid to an acidic peptide can be prevented in the presence of aliphatic hydroxycarboxylic acid. In addition, aliphatic hydroxycarboxylic acid does not inhibit adsorption of a phosphorylated peptide and a phosphoric acid group in the phosphorylated peptide to a metal oxide.

### TECHNICAL FIELD

The present invention relates to a method of separating a phosphorylated peptide or a phosphorylated protein, whereby a phosphorylated protein can be separated from a sample containing a plurality of types of proteins and a phosphorylated peptide can be separated from a sample containing a plurality of types of peptides.

### BACKGROUND ART

There is a series of processes for: cleaving a protein with a digestive enzyme (e.g., trypsin) into peptides; separating the peptides by liquid chromatography; and analyzing the peptides with a mass spectrometer to identify the protein (Non-Patent Document 1). During the processes, a sample comprising cleaved peptides is applied to a metal chelate column so as to concentrate a phosphorylated peptide. Also, in some cases, a sample comprising many protein components is applied to a metal chelate column so as to concentrate a phosphorylated protein.

As described above, upon separation of a phosphorylated peptide and of a phosphorylated protein, chromatography is carried out with the use of a metal chelate column. However, a metal chelate column has low specificity for phosphorylated peptides and for phosphorylated proteins, and thus many acidic peptides are simultaneously concentrated, which is problematic. In order to solve such problem, hitherto, it has been attempted to improve the specificity of a metal chelate column for phosphorylated peptides and for phosphorylated proteins by esterifying a carboxyl group of a peptide. However, since the control of esterification reactions is difficult, such method has not been realized in practice and thus it has not been generally used.

Also, a method of separating a phosphorylated peptide and a phosphorylated protein with the use of a column filled with an oxide such as titanium oxide or zirconium oxide instead of a metal ion has been disclosed (Patent Documents 1 and 2). However, even with the use of such a column filled with an oxide, sufficient levels of specificity of the column for phosphorylated peptides and for phosphorylated proteins cannot be achieved, and thus it is difficult to solve the above problem. Therefore, an attempt to improve the specificity for phosphorylated peptides and for phosphorylated proteins with the use of a salicylic acid derivative as a competing agent for an acidic peptide has been reported (Non-Patent Document 2).

However, the use of a salicylic acid derivative as a competing agent causes the following problems. Firstly, lipophilic properties of a salicylic acid derivative overlap those of a peptide so that a salicylic acid derivative cannot be separated from a phosphorylated peptide by generally used reversed phase chromatography. This problem results in mass spectrometer contamination in a case in which mass spectrometry is conducted after separation. Secondly, although it is certainly possible to improve specificity for a phosphorylated peptide, many non-phosphorylated peptides are simultaneously separated and concentrated, which is also problematic.

Patent Document 1: WO2003/065031

Patent Document 2: JP Patent Publication (Kokai) No. 5-329361 A (1993)

Non-Patent Document 1: Hye Kyong Kweon et al., Analytical Chemistry 78 (6), 1743-1749, 2006

Non-Patent Document 2: Martin R. Larsen et al., Molecular & Cellular Proteomics 4.7 pp. 873-886, 2005

### DISCLOSURE OF THE INVENTION

In view of the above circumstances, it is an objective of the present invention to provide a method whereby a phosphorylated peptide and/or a phosphorylated protein can be specifically separated.

In order to achieve the above objective, the present inventors have conducted intensive studies. As a result, they have found a substance that prevents adsorption of carboxylic acid in an acidic peptide and does not inhibit adsorption of a phosphorylated peptide and a phosphoric acid group in the phosphorylated peptide upon separation of a phosphorylated peptide and/or a phosphorylated peptide with the use of a separation unit filled with a metal oxide. This has led to the completion of the present invention.

Specifically, the present invention encompasses the following.

(1) A method of separating a phosphorylated peptide or a phosphorylated protein, comprising the step of supplying a sample containing a phosphorylated peptide and/or a phosphorylated protein to a separation unit filled with a metal oxide in the presence of an aliphatic hydroxycarboxylic acid.
(2) The method of separating a phosphorylated peptide or a phosphorylated protein according to (1), wherein the aliphatic hydroxycarboxylic acid is a-hydroxycarboxylic acid.
(3) The method of separating a phosphorylated peptide or a phosphorylated protein according to (1), further comprising the step of separating the phosphorylated peptide and the phosphorylated protein from the aliphatic hydroxycarboxylic acid by subjecting a solution eluted from the separation unit to reversed phase chromatography.
(4) The method of separating a phosphorylated peptide or a phosphorylated protein according to (1), wherein the a-hydroxycarboxylic acid is hydrophilic.
(5) The method of separating a phosphorylated peptide or a phosphorylated protein according to (1), wherein the metal oxide is at least one member selected from the group consisting of titanium oxide, zirconium oxide, aluminium oxide, and silicon dioxide.
(6) The method of separating a phosphorylated peptide or a phosphorylated protein according to (1), wherein the metal oxide has a continuous porous structure.
(7) The method of separating a phosphorylated peptide or a phosphorylated protein according to (1), wherein the metal oxide comprises an anatase crystal and/or an amorphous crystal and undergoes a weight reduction of 3 to 70 mg/g during a process of increasing the temperature by 40° C. per minute to 800° C. following heating at 130° C. for 15 minutes upon differential thermogravimetric analysis.
(8) The method of separating a phosphorylated peptide or a phosphorylated protein according to (7), wherein the weight reduction is 4 to 20 mg/g.
(9) A method of mass spectrometry of a phosphorylated peptide and/or a phosphorylated protein, comprising the steps of: supplying a sample containing a phosphorylated peptide and/or a phosphorylated protein separated by the method of separating a phosphorylated peptide or a phosphorylated protein according to any one of (1) to (8) to a mass spectrometer; and carrying out mass measurement of the separated phosphorylated peptide and/or the phosphorylated protein.

In order to achieve the above objective, the present inventors have conducted intensive studies. As a result, they have found that the efficiency of separation of a phosphorylated peptide and/or a phosphorylated protein can be improved using titanium oxide, which is a metal oxide having characteristic physical properties, upon separation of a phosphorylated peptide and/or a phosphorylated peptide with the use of a separation unit filled with a metal oxide. This has led to the completion of the present invention.

Specifically, the present invention encompasses the following.

(10) A method of separating a phosphorylated peptide or a phosphorylated protein, comprising the step of supplying a sample containing a phosphorylated peptide and/or a phosphorylated protein to a separation unit filled with titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis.
(11) The method of separating a phosphorylated peptide or a phosphorylated protein according to (10), wherein the titanium oxide undergoes a weight reduction of 4 to 20 mg/g.
(12) The method of separating a phosphorylated peptide or a phosphorylated protein according to (10), wherein the sample is supplied to the separation unit in the presence of an aliphatic hydroxycarboxylic acid.
(13) The method of separating a phosphorylated peptide or a phosphorylated protein according to (10), wherein the titanium oxide has a continuous porous structure.
(14) A chromatography stationary phase mainly consisting of titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis.
(15) The chromatography stationary phase according to (14), wherein the weight reduction is 4 to 20 mg/g.
(16) The chromatography stationary phase according to (14), wherein the titanium oxide has a continuous porous structure.

This description includes part or all of the contents as disclosed in the description and/or drawings of Japanese Patent Application No. 2006-222316, which is a priority document of the present application.

### BRIEF DESCRIPTION OF THE DRAWINGS

FIG. 1a is a chromatogram showing the measurement results for a sample subjected to trypsin digestion treatment in a chelate-free system.

FIG. 1b is a chromatogram showing the measurement results for a sample subjected to trypsin digestion treatment in a system to which lactic acid serving as a chelate was added.

FIG. 1c is an MS spectrum showing the measurement results for MS spectral intensity at a retention time of 33.6 minutes in the chromatogram shown in FIG. 1b.

FIG. 1d shows an MS/MS spectrum showing the measurement results for MS/MS spectral intensity of a peak with an m/z value of 830.7 in the MS spectrum shown in FIG. 1d.

FIG. 2a is a chromatogram showing the retention time measurement results for malic acid upon LC-MS.

FIG. 2b is a chromatogram showing the retention time measurement results for tartaric acid upon LC-MS.

FIG. 2c is a chromatogram showing the retention time measurement results for citric acid upon LC-MS.

FIG. 2d is a chromatogram showing retention time measurement results for 2,5-dihydroxybenzoic acid upon LC-MS.

FIG. 3 is an SDS-PAGE image showing the results of an experimental example (Example 3) in which a non-phosphorylated protein and a phosphorylated protein were separated and concentrated.

FIG. 4 is an image of a phosphorylated peptide concentration tip having a C2-titania-C2 structure that was produced in Example 5.

FIG. 5 is a characteristic chart showing TG-DTA curves obtained as the result of thermal analysis of the titanium oxide used in Example 6 with the use of a TG-DTA apparatus.

FIG. 6 is a characteristic chart with the horizontal axis representing weight reduction and the vertical axis representing phosphorylated peptide concentration rate. The chart shows the plotting results corresponding to the results listed in table 6.

### BEST MODE FOR CARRYING OUT THE INVENTION

Hereafter, the present invention is described in greater detail with reference to the drawings.

The method of separating a phosphorylated peptide and/or a phosphorylated protein of the present invention is a method wherein a phosphorylated peptide and/or a phosphorylated protein contained in a sample are separated from the other components so as to be concentrated. Herein, such a “sample” is not particularly limited as long as it has a composition comprising a phosphorylated peptide or a phosphorylated protein. Examples thereof include a solution containing a plurality of types of proteins, a solution containing peptides obtained by treating a single protein or a plurality of types of proteins with a digestive enzyme, and a solution containing a plurality of proteins and peptides. In addition, a cell extract obtained by extracting protein components from culture cells or the like or a tissue extract obtained by extracting protein components from tissue collected from an animal individual such as a human can be directly used as such sample.

In a case in which a state of phosphorylation of a specific protein is determined in a more specific manner, a solution obtained by treating the protein with a digestive enzyme such as trypsin can be used. The use of such solution will be described below in greater detail. A phosphorylated peptide can be selectively separated from a group of peptides treated with trypsin by applying the method of separating a phosphorylated peptide and/or a phosphorylated protein of the present invention to a solution obtained as described above, followed by concentration.

In addition, according to the method of separating a phosphorylated peptide and/or a phosphorylated protein of the present invention, a protein and a peptide are not limited, and thus proteins and peptides derived from any types of cells can be targets to be separated. Further, the isoelectric point of a protein is not limited, and thus a protein with any isoelectric point can be a target to be separated.

### First Embodiment

According to the method of separating a phosphorylated peptide and/or a phosphorylated protein of the present invention, when a sample containing a phosphorylated peptide and/or a phosphorylated protein is supplied to a separation unit filled with a metal oxide, an aliphatic hydroxycarboxylic acid is allowed to be present therein. Such an aliphatic hydroxycarboxylic acid may be added to the sample in a preliminary step or it may be independently supplied to a separation unit before supplying the sample to the separation unit. In addition, it is preferable that an aliphatic hydroxycarboxylic acid is added to the sample in a preliminary step and that it also is independently supplied to a separation unit before supply of the sample to the separation unit.

The term “aliphatic hydroxycarboxylic acid” used herein refers to a hydroxycarboxylic acid having an aliphatic skeleton. In some cases, it can include a hydroxycarboxylic acid with a skeleton that does not comprise an aromatic ring. The hydroxycarboxylic acid used herein is preferably an α-hydroxycarboxylic acid; however, it may be a hydroxycarboxylic acid having a hydroxyl group at the β position or γ position.

Specific examples of an aliphatic hydroxycarboxylic acid include α-hydroxycarboxylic acids such as glycolic acid, lactic acid, malic acid, tartaric acid, and citric acid. In addition, an optical isomer of an α-hydroxycarboxylic acid might be present. In such case, either one of the two enantiomers can be used, or a mixture of both enantiomers (e.g., a racemic mixture) can be used according to the method of separating a phosphorylated peptide and/or a phosphorylated protein of the present invention. In addition, a β-hydroxycarboxylic acid such as β-hydroxypropanoic acid can be used as an aliphatic hydroxycarboxylic acid. Further, the compounds specifically described above may be used alone as an aliphatic hydroxycarboxylic acid. Alternatively, a mixture of a plurality of types the compounds may be used as an aliphatic hydroxycarboxylic acid.

According to the method of separating a phosphorylated peptide and/or a phosphorylated protein of the present invention, a separation unit refers to an apparatus capable of: being filled with a metal oxide; selectively retaining a phosphorylated peptide and/or a phosphorylated protein contained in a sample when the sample is supplied to the portion filled with a metal oxide; and separating a phosphorylated peptide and/or a phosphorylated protein from acidic peptides and the like. An example of the separation unit that can be used is a separation column for chromatography. Such a separation column is composed of a tubular member having an inlet and an elution port such that the inside of the tubular member can be filled with a metal oxide. A separation column is not limited at all in terms of shape, size, or material.

Regarding a metal oxide used for a separation unit, the term “metal oxide” used herein includes any substance known to have an affinity to either or both of a phosphorylated peptide and a phosphorylated protein. In particular, examples of such metal oxide include titanium oxide, zirconium oxide, aluminium oxide, aluminium hydroxide, boehmite, and silicon dioxide. According to the method of separating a phosphorylated peptide and/or a phosphorylated protein of the present invention, such metal oxides may be used alone or in a combination of a plurality of types thereof. In particular, regarding a metal oxide, it is preferable that titanium oxide and zirconium oxide be used alone or in combination because of their high affinities to a phosphorylated peptide and/or a phosphorylated protein.

For a method of producing such a metal oxide, a conventionally known method can be used. In addition, when a separation unit is filled with a metal oxide, a variety of ion exchange resins, inorganic ion exchangers, resins, active carbon products, and an argillaceous compound such as montmorillonite, can be used as carriers when a separation unit is filled with a metal oxide.

In particular, a metal oxide used for a separation unit can mainly consist of a metal oxide having a monolithic structure. The term “monolithic structure” used herein refers to a structure composed of a three-dimensional network skeleton and gaps (called macropores or through pores) formed within the skeleton. In other words, the monolithic structure also refers to a continuous porous structure composed of such gaps. In addition, the skeleton constituting a monolithic structure may be made of a material having pores with diameters of several tens of nanometers (called mesopores) or of a material having no such pores. The expression “ . . . mainly consist of a metal oxide having a monolithic structure” indicates that a portion of a metal oxide used for a separation unit may not have a monolithic structure. For example, the expression indicates that a metal oxide in which the monolithic structure is 80%, preferably 90%, and more preferably 95% of the total metal oxide structure is used.

A metal oxide having a monolithic structure can be obtained by a conventionally known method. For instance, titanium oxide having a monolithic structure can be produced by a method disclosed in Junko Konishi et al., “Monolithic TiO₂ with Controlled Multiscale Porosity via a Template-Free Sol-Gel Process Accompanied by Phase Separation” Chem. Mater., Vol. 18, No. 25, 2006. More specifically, a solution containing hydrochloric acid, formamide, and water is added to titanium propoxide (titanium n-propoxide: Ti(OⁿPr)₄) at a freezing temperature with stirring. After stirring for approximately 5 minutes, the uniformly stirred solution is introduced into a test tube, followed by gelatinization at 30° C. The obtained gelatinized substance is allowed to stand at 30° C. to 60° C. for approximately 24 hours. Thereafter, the substance is dried in vacuo at 60° C. for approximately 7 days. Thus, titanium oxide having a monolithic structure can be produced. Also, the gel dried in vacuo may be heat treated at a temperature of approximately 300° C. to 700° C.

Further, a particularly preferable example of a metal oxide used for a separation unit is titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis. Further, it is more preferable to use titanium oxide undergoing such a weight reduction of 4 to 20 mg/g for a separation unit.

The use of titanium oxide undergoing a weight reduction of 3 to 70 mg/g as described above results in further improvement of the ability of a separation unit to retain a phosphorylated peptide and/or a phosphorylated protein. Consequently, the concentration efficiency of a phosphorylated peptide and/or a phosphorylated protein contained in a sample can be improved. In particular, when titanium oxide undergoing a weight reduction of 4 to 20 mg/g as described above is used, the concentration efficiency of a phosphorylated peptide and/or a phosphorylated protein contained in a sample can be further improved.

In such case, the titanium oxide used may comprise both an anatase crystal and an amorphous crystal. Further, the titanium oxide used may consist of an anatase crystal.

Therefore, it is most preferable to use, as a separation unit, titanium oxide comprising an anatase crystal and/or an amorphous crystal and undergoing a weight reduction of 4 to 20 mg/g as described above. When such titanium oxide comprising an anatase crystal and/or an amorphous crystal and undergoing a weight reduction of 4 to 20 mg/g is used as a separation unit, high concentration efficiency can be achieved for a phosphorylated peptide and a phosphorylated protein even with the use of a sample with a complicated composition, such as a cell extract or a tissue extract.

As described above, according to the method of separating a phosphorylated peptide and/or a phosphorylated protein of the present invention, a metal oxide is treated with an aliphatic hydroxycarboxylic acid and then a sample containing a phosphorylated peptide and/or a phosphorylated protein is allowed to come into contact with the metal oxide. Thus, the specificity of a metal oxide for a phosphorylated peptide and for a phosphorylated protein can be further improved. Therefore, according to the method of separating a phosphorylated peptide and/or a phosphorylated protein of the present invention, a phosphorylated peptide and a phosphorylated protein can be efficiently separated from, for example, an acidic peptide that differs from a phosphorylated peptide and/or a phosphorylated protein.

Further, since an aliphatic hydroxycarboxylic acid is a low molecular substance with high hydrophilicity, the elution time therefor does not overlap that for a phosphorylated peptide and/or a phosphorylated protein. Thus, such substance can be removed with a column generally used for reversed phase chromatography. For instance, in a case in which a phosphorylated peptide and/or a phosphorylated protein is separated and then supplied to a mass spectrometer for mass measurement of the phosphorylated peptide or phosphorylated protein, mass spectrometer contamination can be prevented. Therefore, mass measurement of a phosphorylated peptide and a phosphorylated protein can be carried out without mass spectrometer contamination by a series of processes, provided a mass spectrometer via a column for reversed phase chromatography is connected to the rear portion of a separation unit that is used for the method of separating a phosphorylated peptide and/or a phosphorylated protein of the present invention.

In addition, a mass spectrometer is not particularly limited. A mass spectrometer provided based on any principle can be used. In general, a mass spectrometer is composed of a sample injector, an ion source for ionizing a peptide or a protein contained in a sample introduced by the sample injector, an analyzer for separating a peptide or a protein ionized by the ion source, a detector for sensitizing and detecting ions separated in the analyzer, and a data processor for generating a mass spectrum based on the value detected in the detector. It is preferable to use a liquid chromatography column for a sample injector. An ion source is not particularly limited. However, ion sources provided based on principles involving electron ionization, chemical ionization, field desorption, fast atom bombardment, matrix-assisted laser desorption ionization, and electrospray ionization can be used. An analyzer is not particularly limited. However, examples thereof can include a magnetic deflection analyzer, a quadrupole analyzer, an ion trap analyzer, a time-of-flight analyzer, and a Fourier transform ion cyclotron resonance analyzer. Also, a tandem analyzer obtained by combining the above analyzers may be used.

It is particularly preferable to use a mass spectrometer such as an ion trap mass spectrometer or a tandem mass spectrometer for a phosphorylated peptide or a phosphorylated protein separated by the method of separating a phosphorylated peptide and/or a phosphorylated protein of the present invention. This is because even a phosphorylated portion of such a peptide or protein can be identified based on an MS/MS spectrum when an ion trap or tandem mass spectrometer is used.

### Second Embodiment

Further, as described above, the method of separating a phosphorylated peptide and/or a phosphorylated protein of the present invention is not limited to a method wherein a sample is allowed to come into contact with a metal oxide in the presence of an aliphatic hydroxycarboxylic acid. That is, the method of separating a phosphorylated peptide and/or a phosphorylated protein of the present invention may be a method comprising supplying a sample containing a phosphorylated peptide and/or a phosphorylated protein to a separation unit filled with titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis. In other words, a phosphorylated peptide and/or a phosphorylated protein contained in a sample can be efficiently separated with the use of a chromatography apparatus equipped with a stationary phase mainly consisting of titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis.

In such case, it is possible to allow a sample to come into contact with a chromatography stationary phase that has been treated with an aliphatic hydroxycarboxylic acid as described in the first embodiment. However, in this embodiment, it is not essential to cause an aliphatic hydroxycarboxylic acid to come into contact with a chromatography stationary phase. However, when an aliphatic hydroxycarboxylic acid is allowed to come into contact with titanium oxide, effects similar to those described above can be obtained, which is preferable.

As in the case of the first embodiment, the use of the titanium oxide undergoing a weight reduction of 3 to 70 mg/g results in further improvement of the ability of a separation unit to retain a phosphorylated peptide and/or a phosphorylated protein. Consequently, the concentration efficiency of a phosphorylated peptide and/or a phosphorylated protein contained in a sample can be improved. In particular, when the titanium oxide undergoing a weight reduction of 4 to 20 mg/g is used, the concentration efficiency of a phosphorylated peptide and/or a phosphorylated protein contained in a sample can be further improved.

In such case, the titanium oxide used may comprise both an anatase crystal and an amorphous crystal. Further, the titanium oxide used may consist of an anatase crystal.

Therefore, it is most preferable to use the titanium oxide comprising an anatase crystal and/or an amorphous crystal and undergoing a weight reduction of 4 to 20 mg/g for a chromatography stationary phase. When the titanium oxide comprising an anatase crystal and/or an amorphous crystal and undergoing a weight reduction of 4 to 20 mg/g is used as a separation unit, high concentration efficiency can be achieved for a phosphorylated peptide and a phosphorylated protein even with the use of a sample with a complicated composition, such as a cell extract or a tissue extract.

Further, also in this embodiment, titanium oxide having a monolithic structure can be used as the above titanium oxide. Furthermore, in this embodiment, even a phosphorylated portion of a peptide or protein can be identified based on an MS/MS spectrum with the use of an ion trap or tandem mass spectrometer in the cases of a phosphorylated peptide and a phosphorylated protein separated by the method of separating a phosphorylated peptide and/or a phosphorylated protein of the present invention.

The method for separating a phosphorylated peptide and/or a phosphorylated protein of the present invention is hereafter described in greater detail with reference to the following examples, although the technical scope of the present invention is not limited thereto.

### Example 1

In Example 1, experiments for separation and concentration of phosphorylated peptides were conducted with the use of a variety of aliphatic hydroxycarboxylic acids.

First, α-casein (Sigma, Cat. No. C6780), fetuin (Sigma, Cat. No. F2379), and phosvitin (Sigma, Cat. No. P1253) (50 μg each) were separately dissolved in 0.05 M Tris buffer (pH 9.0, Sigma) (20 μL) containing urea (Bio-Rad, Cat. No. 161-0731) (8 M). 1 mg/mL dithiothreitol (Wako Pure Chemical Industries, Ltd. Cat. No. 040-29223: DTT) (1 μL) was added to each resultant, followed by incubation at 37° C. for 30 minutes for reduction of cysteine residues in each protein. Then, 5 mg/mL iodacetamide (Wako Pure Chemical Industries, Ltd. Cat. No. 091-02153) (1 μL) was added to each resultant, followed by incubation at 37° C. for 30 minutes for alkylation of the cysteine residues. 1 mg/mL Lys-C (Wako Pure Chemical Industries, Ltd. Cat No. 125-05061) (1 μL) was added to each resultant, followed by incubation at 37° C. for 4 hours for digestion of each protein.

Next, a 50 mM ammonium bicarbonate buffer (80 mL) and 1 mg/mL trypsin (Promega, Cat. No. V511C) (1 μL) were added to each resultant in that order, followed by incubation at 37° C. for 10 hours for further digestion of Lys-C-digested peptides and undigested portions of each protein. After digestion, a 1% trifluoroacetic acid (TFA) aqueous solution (10 μL) was added to each resultant for deactivation of trypsin. Subsequently, the obtained solutions subjected to digestion treatment were each desalted with the use of an Empore C18-HD disk cartridge (3M) that had been previously washed with acetonitrile and then subjected to conditioning with a 0.1% TFA (trifluoroacetic acid) aqueous solution. Thereafter, centrifugal concentration was conducted for redissolution in 0.1% TFA water containing 5% acetonitrile (100 μL). The solutions (three different solutions) obtained above were mixed together in equal volumes. The resulting solution was designated as a phosphorylated peptide concentration experiment sample solution sample solution.

Next, a 200-μL pipette tip and an Empore C8 disk were used to produce a C8-StageTip (self-made product; J. Rappsilber, Y. Ishihama, M. Mann, Anal Chem 75 (2003) 663). The top portion of the product was filled with 3 mg of titania (titansphere (GL Sciences Inc., Tokyo, Japan)) or zirconia (Zirchrom-PHASE (Zirchrom, Anoka, Minn., USA)) so as to construct a separation column. In addition, a variety of hydroxycarboxylic acids listed in table 1 were each separately dissolved to a concentration of 300 mg/mL in an aqueous solution containing 80% acetonitrile and 0.1% TFA. Thus, solutions A were prepared. A separation column was washed with a different solution A (20 μL). Then, the solution A (100 μL) was mixed with a phosphorylated peptide concentration experiment sample solution (15 μL) containing a peptide mixture in an amount equivalent to 2.5 μg of each protein. The separation column was loaded with the mixture. Thereafter, the separation column was washed with the solution A (20 μL) and an aqueous solution containing 80% acetonitrile and 0.1% TFA (20μ), and the column was loaded with 0.5% ammonia water (40 μL) for phosphorylated peptide elution. Subsequently, the obtained eluate was subjected to centrifugal concentration, followed by dissolution with an aqueous solution (10 μL) containing 1% TFA and 5% acetonitrile. Each LC-MS sample solution was obtained as above.

Next, the obtained LC-MS sample solutions were subjected to measurement with an LC (C18 column)/MS (Applied Biosystems/MDS-Sciex QSTAR XL) system. HPLC conditions are described below. A self-made electrospray-equipped column (Y. Ishihama, J. Rappsilber, J. S. Andersen, M. Mann, J Chromatogr A 979 (2002) 233) (0.1×150 mm) filled with C18 silica gel (ReproSil-Pur 120 C18-AQ; 3 μm) was used. 0.5% acetic acid water was used as a mobile phase A. 0.5% acetic acid water containing 80% acetonitrile was used as a mobile phase B. The initial concentration of B was 5%. The concentration B was linearly increased to 30% during the first 15 minutes and to 100% during the following 5 minutes. Then, the mobile phase B concentration was maintained at 100% for 5 minutes. Thereafter, the mobile phase B concentration was decreased to 5%. 35 minutes later, the next sample was introduced into the column. An Agilent 1100 nanopump (Agilent Technologies) was used for liquid feeding, and analysis was carried out at a flow rate of 500 nL/min. Each LC-MS sample solution (5 μL) was fed with the use of an autosampler PAL (CTC) so as to be first introduced into a sample loop of an injector and then delivered to an analysis column. A custom-ordered column holder (produced by Nikkyo Technos Co., Ltd.) was mounted on QSTAR XL (Applied Biosystems/MDS-Sciex) equipped with an XYZ stage (Proxeon) in a manner such that the position of the electrospray-equipped column was allowed to be controlled in an arbitrary manner. An ESI voltage of 2.4 kV was applied to the column via a metal connector (Varco) provided to the pump side of the column. For measurement, a 1-second survey scan with an information-dependent acquisition mode and then a maximum of three MSMS scans (0.6 second each) were carried out. Switching from the MSMS scan mode to the survey scan mode took place for every single spectrum.

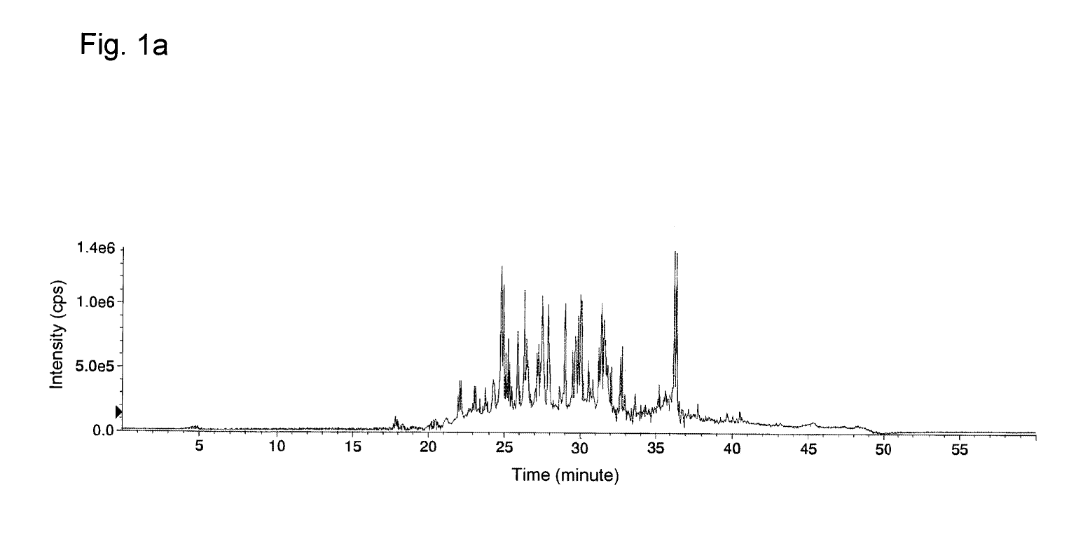

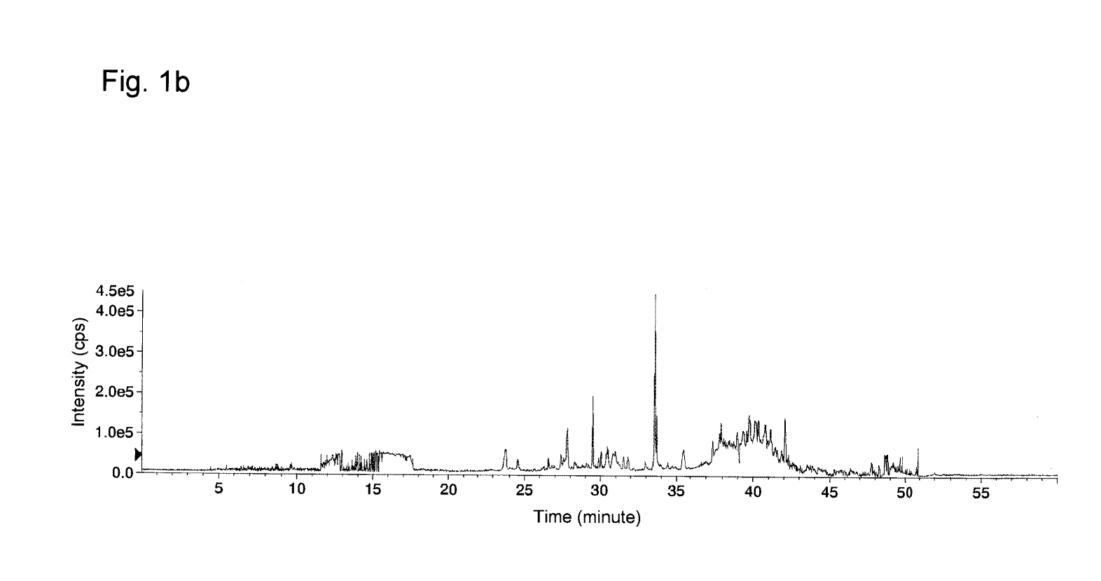

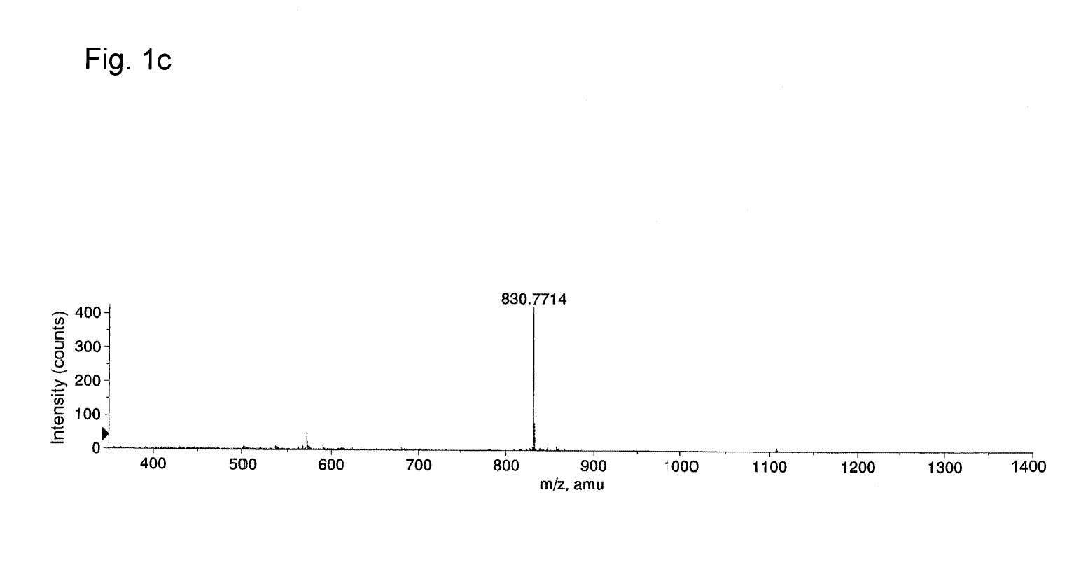

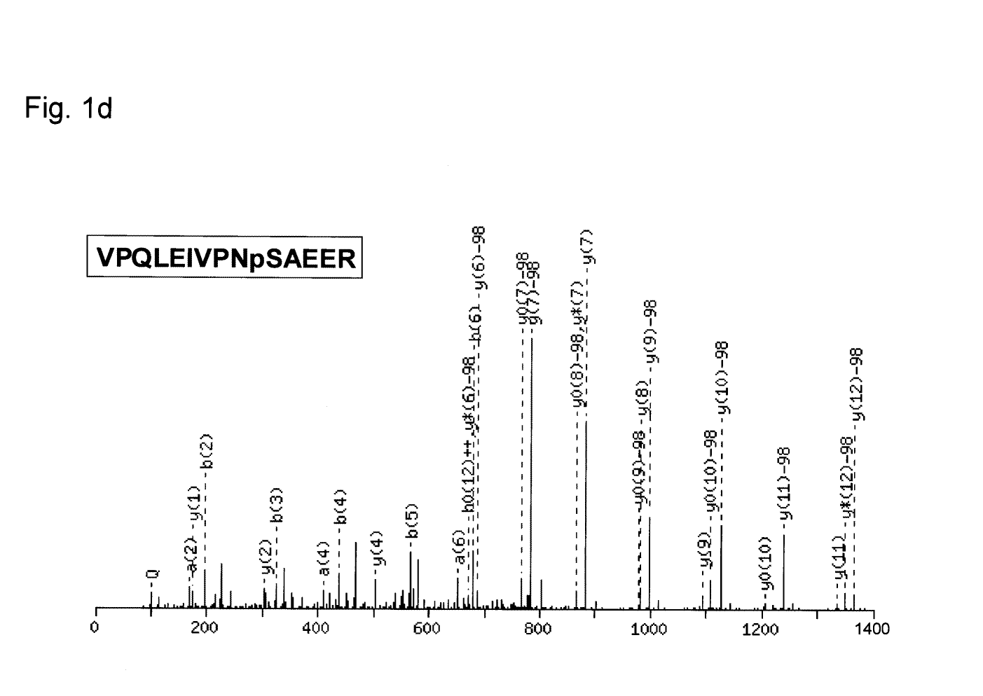

For the data obtained, automatic protein identification was carried out using Mascot (Matrix science) and the Swiss-Prot database. Quantification for a desired peak was carried out using Analyst QS v1.1 (AB). Table 1 shows the results. In addition, FIG. 1 shows a typical example of phosphorylated peptide identification. In addition, FIG. 1a shows measurement results for a chelate-free system. FIG. 1b shows measurement results for a system to which lactic acid serving as a chelate was added.

TABLE 1

| | | | Number of | Number of | | | | | |
|---|---|---|---|---|---|---|---|---|---|
| | | | non-phosphorylated | phosphorylated | Peak area (counts)*¹ | | | | |
| Metal | | MS/MS | peptides identified | peptides identified | CDSSPDp | DIGpSEpST | VPQLEIVP | TVDMEpS | TTpSFPHA |
| oxide | Chelate | number | (Score > 30) | (Score > 30) | SAEDVRK | EDQAMEDIK | NpSAEER | TEVFTK | pSAAEGER |
| ZrO₂ | Glycolic acid | 64 | 4 | 8 | 1350 | 12 | 12600 | 19500 | 3450 |
| ZrO₂ | Lactic acid | 89 | 1 | 10 | 4160 | 749 | 15600 | 22400 | 15900 |
| ZrO₂ | Malic acid | 104 | 0 | 6 | 918 | 22 | 37300 | 6580 | 2100 |
| ZrO₂ | Tartaric acid | 55 | 3 | 5 | 66 | 2 | 16000 | 5650 | 482 |
| ZrO₂ | Citric acid | 25 | 0 | 4 | 154 | 10 | 9640 | 2490 | 1200 |
| ZrO₂ | L-lactic acid | 51 | 1 | 8 | 1430 | 70 | 23100 | 13700 | 2750 |
| ZrO₂ | β-HPA*² | 78 | 0 | 11 | 5310 | 4080 | 19200 | 13400 | 15800 |
| ZrO₂ | 2,5-DHB*³ | 64 | 3 | 3 | 96 | 196 | 18400 | 253 | 245 |
| ZrO₂ | None | 329 | 13 | 9 | 4950 | 2180 | 44200 | 2750 | 5350 |
| TiO₂ | Glycolic acid | 123 | 11 | 10 | 5580 | 324 | 37800 | 26100 | 20600 |
| TiO₂ | Lactic acid | 76 | 2 | 11 | 7440 | 2470 | 79500 | 29500 | 44800 |
| TiO₂ | Malic acid | 31 | 0 | 6 | 1820 | 358 | 11900 | 12900 | 128 |
| TiO₂ | Tartaric acid | 55 | 2 | 8 | 279 | 41 | 4920 | 13000 | 1210 |
| TiO₂ | Citric acid | 22 | 0 | 5 | 175 | 41 | 14500 | 1620 | 819 |
| TiO₂ | L-lactic acid | 92 | 0 | 12 | 5000 | 2550 | 58600 | 17000 | 8270 |
| TiO₂ | β-HPA*² | 53 | 0 | 6 | 2910 | 568 | 60100 | 6880 | 4940 |
| TiO₂ | 2,5-DHB*³ | 55 | 3 | 4 | 162 | 615 | 84900 | 2750 | 633 |
| TiO₂ | None | 330 | 10 | 8 | 4600 | 1100 | 40200 | 5130 | 9330 |

*¹α-casein, fetuin, phosvitin (1.25 mg of each protein)
*²β-hydroxypropionic acid
*³2,5-dihydroxybenzoic acid

As is apparent from table 1, in each chelate case, the phosphorylated peptide selectivity was improved over that in a case in which a chelate serving as a competitive agent was not added. In addition, in a case involving the use of an aliphatic hydroxycarboxylic acid as a chelate, in addition to the phosphorylated peptide selectivity, the phosphorylated peptide collection rate was improved over that in the case of 2,5-DHB, which is an aromatic hydroxycarboxylic acid (Maetin R. Laesen et al., Molecular & Cellular Proteomics 4.7 pp. 873-886). Particularly in the case involving the use of β-hydroxypropionic acid, which is β-hydroxycarboxylic acid, or lactic acid as an aliphatic hydroxycarboxylic acid, the phosphorylated peptide selectivity and the phosphorylated peptide collection rate were found to be significantly improved.

Herein, the hydroxycarboxylic acid reagents used are as follows.

Glycolic acid: WAKO 071-01512
DL-lactic acid: WAKO 128-00056
L-lactic acid: WAKO 129-02666
Malic acid: WAKO 138-07512
L-tartaric acid: WAKO 203-00052
Citric acid: WAKO 036-05522
β-hydroxypropionic acid (β-HPA): Tokyo Chemical Industry Co., Ltd. H0297
2,5-dihydroxybenzoic acid (2,5-DHB): Aldrich 149357-10G

### Example 2

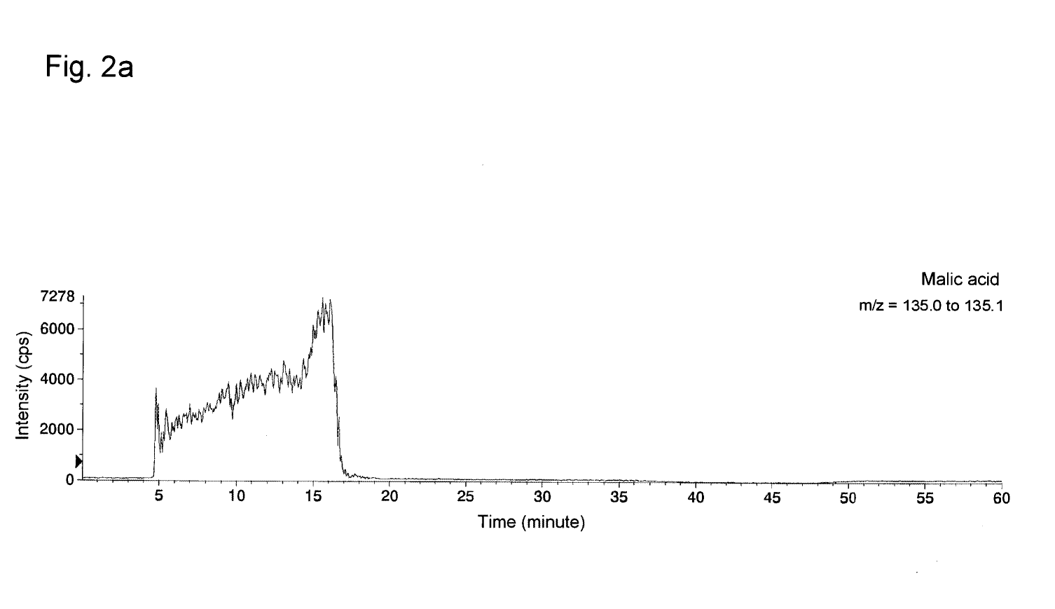

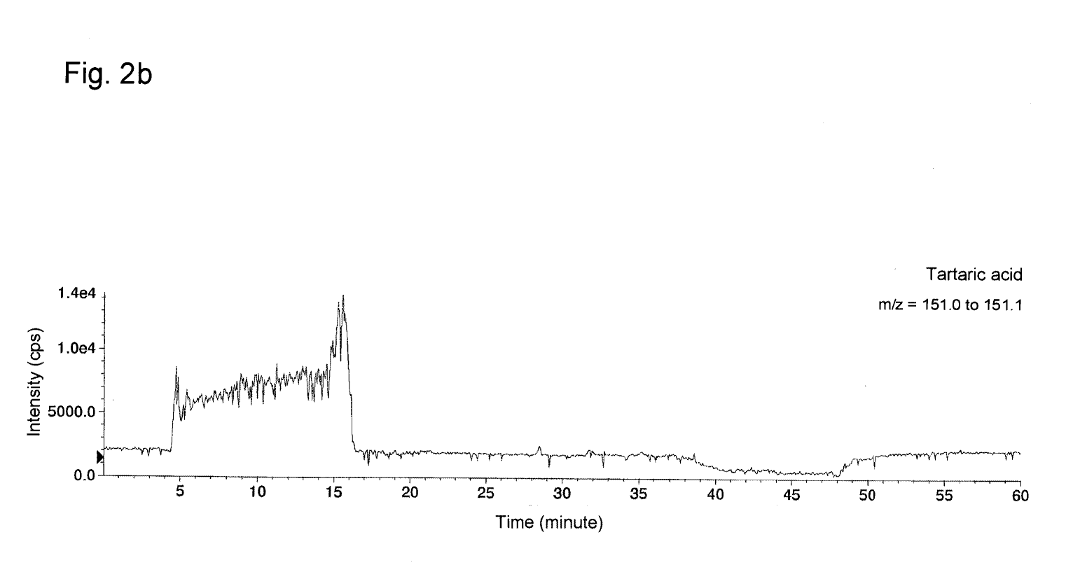

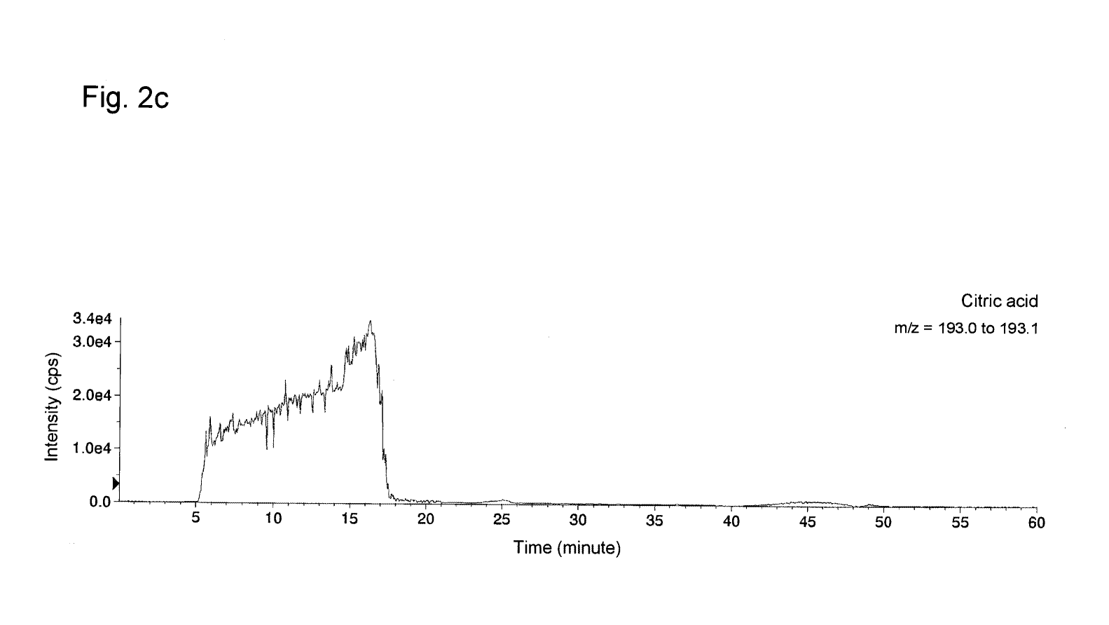

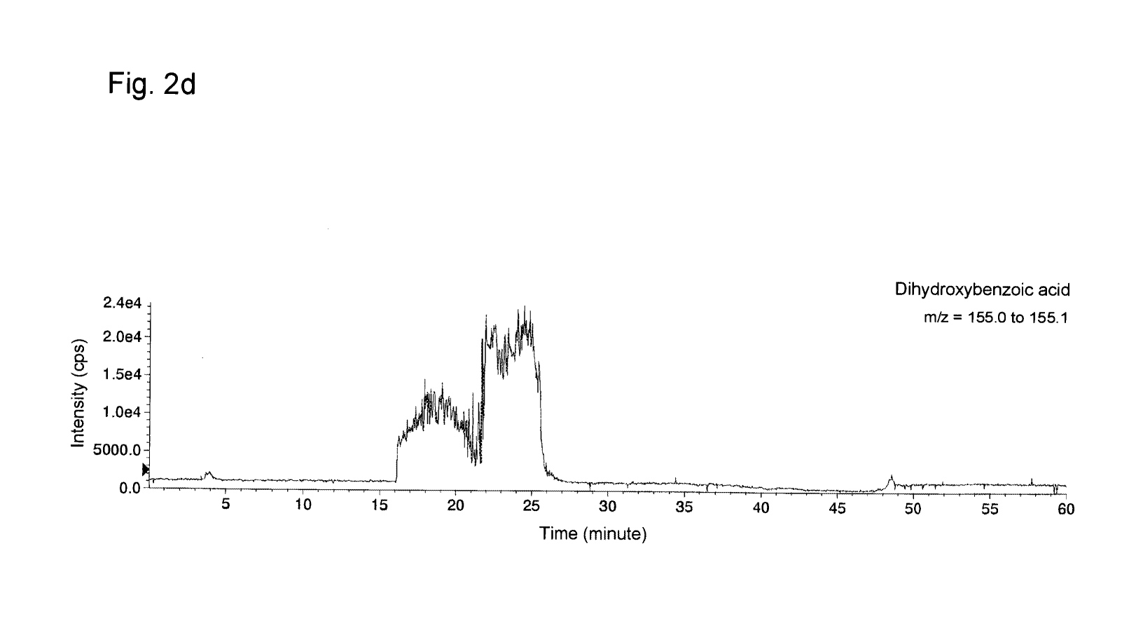

In Example 2, the retention time for a hydroxycarboxylic acid that had been added as a chelate in Example 1 was examined. Specifically, malic acid, tartaric acid, and citric acid, which are aliphatic hydroxycarboxylic acids, were examined in terms of elution time. Also, 2,5-DHB, which is an aromatic hydroxycarboxylic acid, was examined in terms of retention time. FIGS. 2a to 2d each show the retention time for a hydroxycarboxylic acid upon LC-MS. Note that FIGS. 2a to 2d show the retention times for malic acid, tartaric acid, citric acid, and 2,5-DHB.

As is apparent from FIG. 2, upon LC-MS, elution of 2,5-DHB took place within 18 to 35 minutes, which corresponds to the elution time for a trypsin-digested peptide. Meanwhile, it is understood that malic acid, tartaric acid, and citric acid, which are aliphatic hydroxycarboxylic acids, were not retained in C18, as in the case of the sample solvent. The above results revealed that an aliphatic hydroxycarboxylic acid can be removed with a reversed-phase pretreatment column even when used as a chelate. On the other hand, 2,5-DHB was unable to be removed in such a manner, and thus it was thought to cause unstable conditions during mass spectrometry processes with the use of an LC-MS system or the like. Such unstable conditions include column clogging, inhibition of peptide ionization, and sensitivity reduction caused by mass spectrometer contamination.

### Example 3

In Example 3, experiments for separation and concentration of phosphorylated proteins were conducted with the use of a variety of aliphatic hydroxycarboxylic acids.

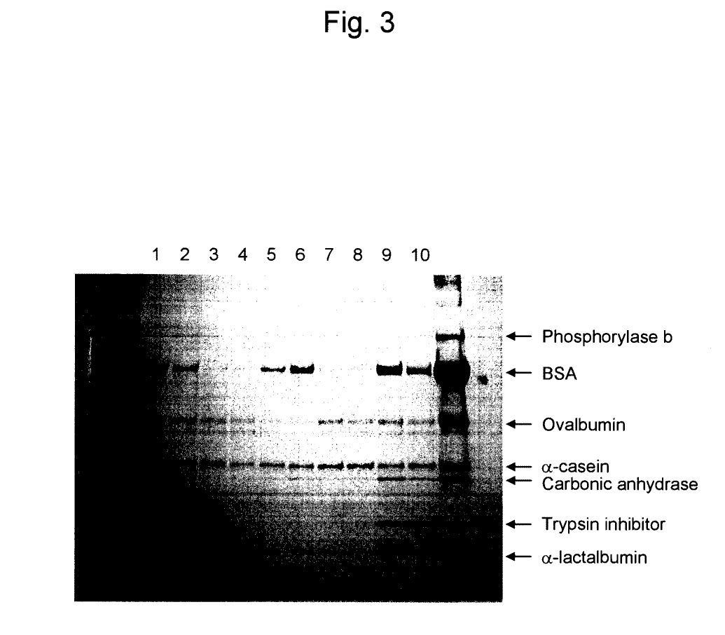

First, 1 mg of bovine serum albumin (BSA) (Wako Pure Chemical Industries, Ltd., Cat. No. 016-15091), which is a non-phosphorylated protein, 0.1 mg of α-casein (SIGMA Cat. No. C6780), which is a phosphorylated protein, and a molecular weight marker kit (GE healthcare Cat. No. 17-0446-01; phosphorylase b (67 μg), BSA (83 μg), obalbumin (147 μg), carbonic anhydrase (83 μg), a trypsin inhibitor (80 μg), and α-lactalbumin (116 μg) per vial) were dissolved in a solution containing 30 mM MES buffer (4-morpholineethanesulfonic acid) (pH=6.0), 8 M urea, and 0.25% CHAPS (3-[(3-cholamidopropyl)dimethylammonio]-1-propanesulfonate) (0.5 mL). Thus, a sample solution (A) was prepared. 3 mg of non-porous zirconia (pore size: 0.5 μm; Zirchrom) was filled in a 1.5-mL tube, followed by washing with a solution containing 30 mM MES buffer (pH=6.0), 8 M urea, and 0.25% CHAPS (50 μL). Then, a solution containing 0.2 M hydroxycarboxylic acid, 30 mM MES buffer (pH=6.0), 8 M urea, and 0.25% CHAPS (25 μL) was mixed with a sample solution (A) (25 μL) and the resultant was added to the tube. After stirring at 37° C. for 30 minutes, centrifugation at 15000 G for 1 minute was carried out. Then, the solution was recovered. The resultant was washed with a solution containing a different 0.2 M aliphatic hydroxycarboxylic acid, 30 mM MES buffer (pH=6.0), 8 M urea, and 0.25% CHAPS (50 μL) and a solution containing 30 mM MES buffer (pH=6.0), 8 M urea, and 0.25% CHAPS (50 μL), followed by suspension in a solution containing 1% ammonia water, 8 M urea, and 0.25% CHAPS (50 μL). The resultant was subjected to stirring at 37° C. for 10 minutes, followed by centrifugation at 15000 G for 1 minute. Then, the solution was recovered for use as a sample solution. Each sample solution was analyzed by SDS-PAGE (4%-20% gradient gel; Daiichi chemical Co., Ltd.: 301506), followed by staining with coomassie brilliant blue (CBB) for detection. FIG. 3 shows the results.

In an SDS-PAGE image shown in FIG. 3, lanes 1 and 2 each represent a sample containing lactic acid serving as an aliphatic hydroxycarboxylic acid, lanes 3 and 4 each represent a sample containing glucuronic acid serving as an aliphatic hydroxycarboxylic acid, lanes 5 and 6 each represent a sample containing glyceric acid hemicalcium hydrate salt serving as an aliphatic hydroxycarboxylic acid, lanes 7 and 8 each represent a sample containing sodium glutamate and potassium aspartate instead of an aliphatic hydroxycarboxylic acid, and lanes 9 and 10 each represent a sample containing no aliphatic hydroxycarboxylic acid.

As is apparent from FIG. 3, compared with the samples containing no aliphatic hydroxycarboxylic acid in lanes 9 and 10, the samples containing different aliphatic hydroxycarboxylic acids in lanes 1 to 6 experienced decreases in BSA, which is a non-phosphorylated protein, carbonic anhydrase, a trypsin inhibitor, and α-lactalbumin. Meanwhile, concentration of α-casein, which is a phosphorylated protein, was possible in lanes 1 to 6. Substantially no non-phosphorylated protein was detected especially in the samples (containing glucuronic acid) in lanes 3 and 4, and thus the samples exhibited high levels of selectivity. The samples containing glucuronic acid serving as an aliphatic hydroxycarboxylic acid (lanes 3 and 4) were compared with the samples containing glutamic acid and aspartic acid, these acids being reported to be used in combination with aluminum hydroxide in order to obtain desired effects (Wolschin, F. et al, Proteomics, 5, 4389-4397, 2005) (lanes 7 and 8). As a result, the BSA removal rate was found to be significantly improved, particularly in the samples containing glucuronic acid, although slightly light ovalbumin bands were observed in lanes 3 and 4.

### Example 4

In this Example, experiments for separation and concentration of phosphorylated proteins were conducted with the use of titanium oxide (hereinafter referred to as titania monolith) having a continuous porous structure.

The sample solution used in this Example was the same as the sample solution used in Example 1. Titania monolith used was a prototype product obtained from GL Sciences Inc. The obtained titania monolith had a surface area of 75.2 m²/g and a pore size of 17.6 nm.

First, titania monolith (1 mg) was filled to a 1.5-mL vial. An aqueous solution (80% acetonitrile and 0.1% TFA) containing 300 mg/mL lactic acid (50 μl) (solution A) was added thereto for dispersion, followed by centrifugal treatment at 2000 g to remove the supernatant. A sample solution (7.5 μL) containing different protein digests (2.5 g each) was added to titania monolith with a solution A (50 μL) for immersion at 25° C. for 20 minutes. Then, centrifugal treatment was conducted at 2000 g to remove the supernatant. The resultant was washed with a solution A and an aqueous solution (80% acetonitrile and 0.1% TFA) (50 μL each), followed by elution twice with 0.5% ammonia water (50 μL). After centrifugal concentration, the resultant was dissolved in an aqueous solution containing 1% TFA and 5% acetonitrile (10 μL). Thus, an LC-MS sample solution was prepared. As a control sample, an LC-MS sample solution was prepared under the same conditions as in Example 1 except that lactic acid was not used.

In this Example, LC-MS assay was conducted under the same conditions as in Example 1. Tables 2 and 3 show the results.

TABLE 2

| | Number of peptides identified (no overlapping) | | | | | |
|---|---|---|---|---|---|---|
| | Addition of lactic acid | | | No addition of lactic acid | | |
| | 1^{st} | 2^{nd} | Average | 1^{st} | 2^{nd} | Average |
| Phosphorylated | 7 | 7 | 7.0 | 6 | 8 | 7.0 |
| peptide | | | | | | |
| Non-phosphorylated | 10 | 6 | 8.0 | 17 | 18 | 17.5 |
| peptide | | | | | | |
| Phosphorylated | 41% | 54% | 47% | 26% | 31% | 29% |
| peptide content | | | | | | |

TABLE 31

| | Total MS chromatogram peak area | | | | | |
|---|---|---|---|---|---|---|
| | Addition of lactic acid | | | No addition of lactic acid | | |
| | 1^{st} | 2^{nd} | Average | 1^{st} | 2^{nd} | Average |
| Phosphorylated | 3400 | 1700 | 2550 | 2800 | 2900 | 2850 |
| peptide | | | | | | |
| Non-phosphorylated | 4800 | 2400 | 3600 | 27900 | 30000 | 28950 |
| peptide | | | | | | |
| Phosphorylated | 42% | 41% | 42% | 9% | 9% | 9% |
| peptide content | | | | | | |

As shown in tables 2 and 3, even in a case in which titania monolith was used as a metal oxide, the phosphorylated peptide content was obviously improved with the addition of lactic acid. Such improvement was significantly observed in relation to MS signal intensity to a greater extent than in relation to the number of peptides identified. The above results revealed that a titania monolith filler has effects of concentrating a phosphorylated peptide as in the case of a particulate filler, and that such effects are further enhanced with the addition of lactic acid.

### Example 5

In this Example, it was examined whether it would be possible to comprehensively analyze phosphorylated peptides contained in a cell extract sample by using the method of the present invention.

First, HeLa cells derived from human cervical cancer were cultured in a 9-cm culture dish by a conventional method. The cells were placed in a Dawn's homogenizer and then homogenized at 10 strokes with the addition of phosphatase inhibitor cocktails 1 and 2 (Sigma; Cat. Nos. P2850 and P5726) and a protease inhibitor (Sigma; Cat. No. P8340), followed by centrifugal treatment at 1,500 g for 10 minutes. Thereafter, the supernatant was collected and subjected to centrifugal concentration. The resultant was dissolved in a 0.05 M Tris buffer (pH 9.0, Sigma) containing urea (Bio-Rad; Cat. No. 161-0731) (8 M) (20 μL). 1 mg/mL dithiothreitol (Wako Pure Chemical Industries, Ltd.; Cat. No. 040-29223: DTT) (1 μL) was added thereto, followed by incubation at 37° C. for 30 minutes for reduction of cysteine residues in each protein. Then, 5 mg/mL iodacetamide (Wako Pure Chemical Industries, Ltd. Cat. No. 091-02153) (1 μL) was added to the resultant, followed by incubation at 37° C. for 30 minutes for alkylation of the cysteine residues. 1 mg/mL Lys-C (Wako Pure Chemical Industries, Ltd. Cat No. 125-05061) (1 μL) was added thereto, followed by incubation at 37° C. for 4 hours for protein digestion. After addition of a 50 mM ammonium bicarbonate buffer (80 mL), 1 mg/mL trypsin (Promega; Cat. No. V511C) (1 μL) was added to the resultant, followed by incubation at 37° C. for 10 hours for digestion of Lys-C-digested peptides and undigested portions of each protein. After digestion, trypsin was deactivated with the addition of a 1% trifluoroacetic acid (TFA) aqueous solution (10 μL). The resultant was desalted with an Empore C18-HD disk cartridge (3M) that had been preliminarily washed with acetonitrile and then subjected to conditioning with a 0.1% TFA aqueous solution. Thus, a sample solution was prepared.

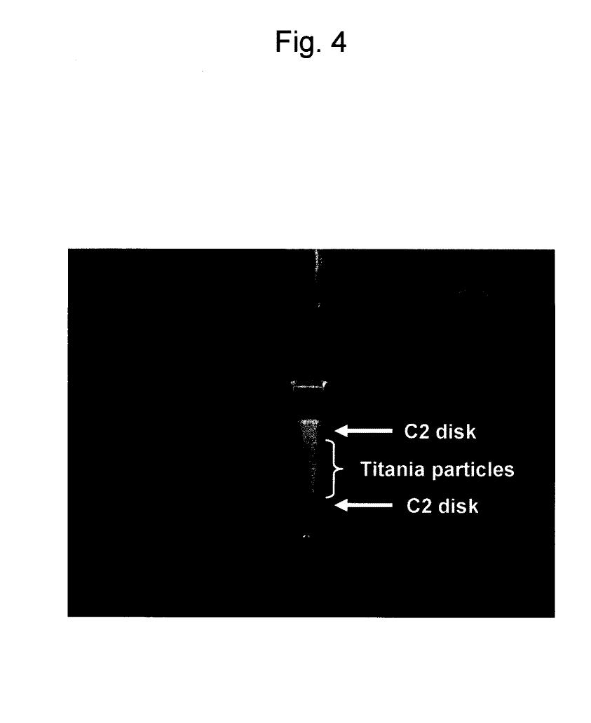

Next, a 10-μL pipette tip and an Empore C2 disk were used to prepare a C2-StageTip (self-made product; J. Rappsilber, Y. Ishihama, M. Mann, Anal Chem 75 (2003) 663), and an upper portion thereof was filled with titania (1 mg). Further, the portion located above the upper portion was filled with an Empore C2 disk such that a phosphorylated peptide concentration tip having a C2-titania-C2 structure was produced (FIG. 4).

Then, DL-lactic acid (Wako Pure Chemical Industries, Ltd.; Cat. No. 128-00056) was dissolved in an aqueous solution containing 80% acetonitrile and 0.1% TFA to a concentration of 300 mg/mL (solution A). A phosphorylated peptide concentration tip was washed with the solution A (20 μL) for conditioning of the tip. A sample solution and the solution A were mixed at a ratio of 1:1 and the phosphorylated peptide concentration tip was loaded with the mixture. The tip was washed with a solution A (20 μL) and an aqueous solution containing 80% acetonitrile and 0.1% TFA, followed by elution with 0.5% ammonia water (50 μL) and centrifugal concentration. Thereafter, the resultant was dissolved in an aqueous solution containing 1% TFA and 5% acetonitrile (10 μL). Thus, an LC-MS sample solution was prepared.

The sample solution was subjected to measurement with an LC (C18 column)/MS (ThermoFisher LTQ-orbitrap) system. HPLC conditions are described below. A self-made electrospray-equipped column (Y. Ishihama, J. Rappsilber, J. S. Andersen, M. Mann, J Chromatogr A 979 (2002) 233) (0.1×150 mm) filled with C18 silica gel (ReproSil-Pur 120 C18-AQ, 3 μm) was used. 0.5% acetic acid water was used as a mobile phase A. 0.5% acetic acid water containing 80% acetonitrile was used as a mobile phase B. The initial concentration of B was 5%. The concentration B was linearly increased to 10% during the first 5 minutes and to 40% during the following 60 minutes. Further, it was linearly increased to 100% for 5 minutes. Then, the mobile phase B concentration was maintained at 100% for 10 minutes. Thereafter, the mobile phase B concentration was decreased to 5%. 30 minutes later, the next sample was introduced into the column. An Ultimate3000 system (Dionex Corporation) was used for liquid feeding, followed by analysis at a flow rate of 500 nL/min. The LC-MS sample solution (5 μL) was fed with the use of an autosampler HTC-PAL (CTC) so as to be first introduced into a sample loop of an injector and then delivered to an analysis column. An electrospray-equipped column was mounted on a nano LC-MS interface (Nikkyo Technos Co., Ltd.). An ESI voltage of 2.4 kV was applied to the column via a metal connector (Varco) provided to the pump side of the column. For measurement, a survey scan using an orbitrap with a data-dependent mode and then a maximum of ten MSMS scans with an ion trap were carried out. Switching from the MSMS scan mode to the survey scan mode took place for every single spectrum.

For the data obtained, peptide identification was carried out using Mascot (Matrix science) and the Swiss-Prot database. Quantification of target peaks was carried out using Mass Navigator v1.2 developed by MITSUI KNOWLEDGE INDUSTRY CO., LTD. Table 4 shows the results.

TABLE 4

| | Number of peptides | | | Total MS chromatogram | | |
|---|---|---|---|---|---|---|
| | identified (no overlapping) | | | peak area | | |
| | 1^{st} | 2^{nd} | Average | 1^{st} | 2^{nd} | Average |
| Phosphorylated | 625 | 559 | 592 | 8655 | 6493 | 7574 |
| peptide | | | | | | |
| Non-phosphorylated | 66 | 75 | 70.5 | 270 | 219 | 245 |
| peptide | | | | | | |
| Phosphorylated | 90.4% | 88.2% | 89.4% | 97.0% | 96.7% | 96.9% |
| peptide content | | | | | | |

It has been found that phosphorylated peptides can be concentrated directly even from a mixed sample with a complicated composition, such as a cell extract, without prefractionation by applying the method of the present invention in the manner described above. Specifically, approximately 600 peptides each having a unique sequence can be identified by a single instance of LC-MS analysis. In addition, the peptide content was approximately 90%. In addition, when the concentration efficiency was calculated based on MS signal intensity rather than the number of peptides, the phosphorylated peptide content was approximately 97%, indicating that phosphorylated peptides can be concentrated with very high selectivity.

### Example 6

In this Example, experiments for separation and concentration of phosphorylated peptides were conducted with the use of a variety of titanium oxides. The 13 types of titanium oxides listed in table 5 were used in this Example.

TABLE 5

| Sample name | Origin |
|---|---|
| Sachtopore-NP | Diameter: 5 μm; pore size: 300 Å; Zirchrom Separation |
| Wako-anatase | Wako Pure Chemical Industries, Ltd.; Cat. No 207-11121 |
| YU-200-1 | Self-made synthesized product calcinated at 200° C.; Lot 1 |
| YU-200-2 | Self-made synthesized product calcinated at 200° C.; Lot 2 |
| YU-500 | Self-made synthesized product calcinated at 500° C. |
| Titansphere | Titansphere (Lot Ti-213, GL Sciences Inc.) |
| Titansphere-S | Alkali-washed Titansphere product |
| GLP-000 | Prototype (non-calcinated particle type); GL Sciences Inc. |
| GLP-200 | Prototype (200° C.-calcinated particle type); GL Sciences Inc. |
| GLP-300 | Prototype (300° C.-calcinated particle type); GL Sciences Inc. |
| GLP-400 | Prototype (400° C.-calcinated particle type); GL Sciences Inc. |
| GLP-500 | Prototype (500° C.-calcinated particle type); GL Sciences Inc. |
| GLM-500 | Prototype (500° C.-calcinated monolith type); GL Sciences Inc. |

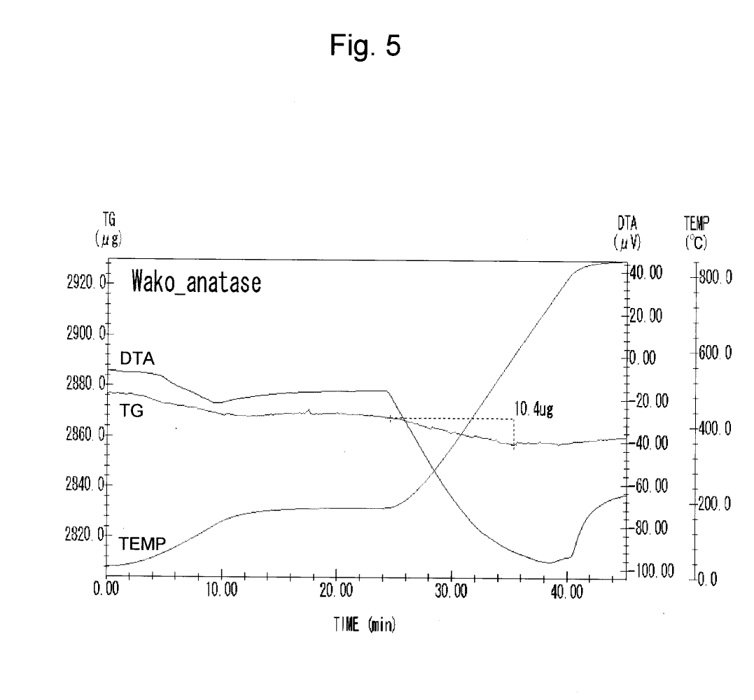

The 13 above types of titanium oxides were subjected to thermal analysis with the use of a TG-DTA apparatus (system WS002, MacScience). Upon thermal analysis, each sample was weighed in an amount of several milligrams. The temperature was increased by 20° C. per minute to 130° C. in a nitrogen atmosphere, retained for 15 minutes, increased by 40° C. per minute to 800° C., and retained for 10 minutes. FIG. 5 shows examples of the obtained TG-DTA curves.

Each titanium oxide sample was subjected to the above thermal analysis. The maximum weight reduction that took place after the time point at 130° C. was determined during the step of increasing the temperature from 130° C. to 800° C. Then, the weight reduction per unit weight of the sample weighed was calculated.

In addition to thermal analysis, phosphorylated peptides were concentrated under the same conditions as in Example 1, except that the above titanium oxides were used. Then, the phosphorylated peptide concentration rate (%) was calculated by the following equation: phosphorylated peptide concentration rate (%)=(total peak area for phosphorylated peptides)/(total peak area for peptides)×100. Table 6 shows the results. In addition, regarding crystal shapes, the results listed in table 6 were evaluated based on powder X-ray patterns.

TABLE 6

| | Weight | | Phosphorylated |
|---|---|---|---|
| | reduction per | | peptide |
| | unit sample | | concentration |
| Sample name | weight (mg/g) | Crystal shape* | rate (%) |
| Sachtopore-NP | 3.1 | Anatase | 35.1 |
| GLM-500 | 4.2 | Anatase | 41.4 |
| Titansphere-S | 4.6 | Anatase + amorphous | 93.9 |
| YU-500 | 4.8 | Anatase + amorphous | 74.1 |
| Wako-anatase | 6.8 | Anatase | 26.2 |
| Titansphere | 8.4 | Anatase + amorphous | 96.2 |
| GLP-400 | 9.3 | Anatase + amorphous | 90.6 |
| GLP-500 | 10.1 | Anatase + amorphous | 95.7 |
| YU-200-1 | 16.1 | Anatase + amorphous | 79.5 |
| YU-200-2 | 27.7 | Anatase + amorphous | 24.0 |
| GLP-300 | 29.8 | Anatase + amorphous | 59.6 |
| GLP-200 | 75.6 | Anatase + amorphous | 16.6 |
| GLP-000 | 77.0 | Anatase + amorphous | 5.3 |

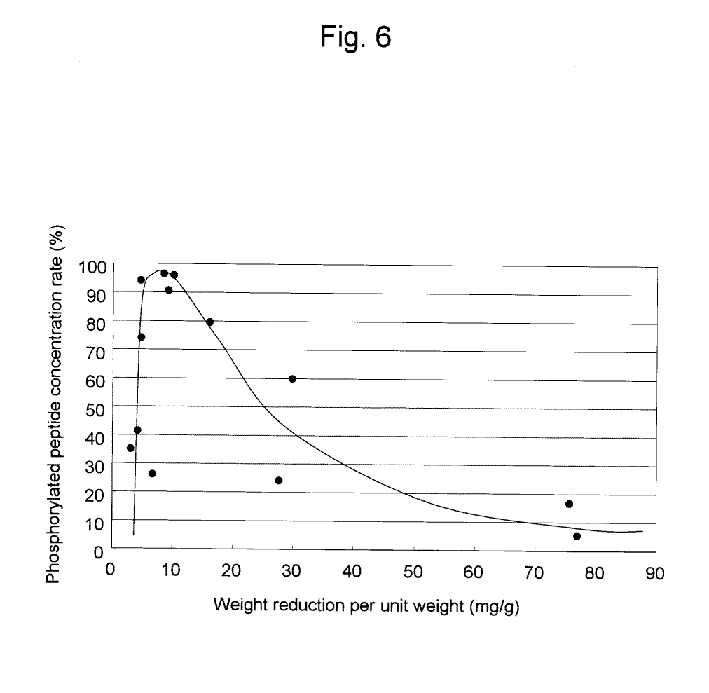

FIG. 6 is a chart with the horizontal axis representing weight reduction and the vertical axis representing phosphorylated peptide concentration rate. The chart shows the plotting results corresponding to the results listed in table 6. As shown in table 6 and FIG. 6, it has been found that high phosphorylated peptide concentration efficiencies can be obtained by selecting a titanium oxide comprising an anatase crystal or a combination of an anatase crystal and an amorphous or different crystal undergoing a weight reduction per unit weight of titanium oxide of preferably 3 to 70 mg/g and more preferably 4.5 to 20 mg/g at 130° C. or more upon thermal analysis.

### INDUSTRIAL APPLICABILITY

According to the present invention, a novel method of separating a phosphorylated peptide or a phosphorylated protein, whereby a phosphorylated peptide and/or a phosphorylated protein contained in a sample can be specifically separated, can be provided. According to the method of separating a phosphorylated peptide or a phosphorylated protein of the present invention, a phosphorylated peptide or a phosphorylated protein can be separated with high selectivity by removing acidic peptides. In addition, according to the method of separating a phosphorylated peptide or a phosphorylated protein of the present invention, since an aliphatic hydroxycarboxylic acid is a low molecular substance with high hydrophilicity, it can be readily separated from a phosphorylated peptide and a phosphorylated protein that are intended to be separated. Therefore, according to the method of separating a phosphorylated peptide or a phosphorylated protein of the present invention, a sample containing a phosphorylated peptide or a phosphorylated protein that has been separated can be directly applied to a mass spectrometer and the like.

All publications, patents, and patent applications cited herein are incorporated herein by reference in their entirety.

1. A method of separating a phosphorylated peptide or a phosphorylated protein, comprising the step of supplying a sample containing a phosphorylated peptide and/or a phosphorylated protein to a separation unit filled with a metal oxide in the presence of an aliphatic hydroxycarboxylic acid.

2. The method of separating a phosphorylated peptide or a phosphorylated protein according to claim 1, wherein the aliphatic hydroxycarboxylic acid is α-hydroxycarboxylic acid.

3. The method of separating a phosphorylated peptide or a phosphorylated protein according to claim 1, further comprising the step of separating the phosphorylated peptide and the phosphorylated protein from the aliphatic hydroxycarboxylic acid by subjecting a solution eluted from the separation unit to reversed phase chromatography.

4. The method of separating a phosphorylated peptide or a phosphorylated protein according to claim 1, wherein the α-hydroxycarboxylic acid is hydrophilic.

5. The method of separating a phosphorylated peptide or a phosphorylated protein according to claim 1, wherein the metal oxide is at least one member selected from the group consisting of titanium oxide, zirconium oxide, aluminium oxide, aluminum hydroxide, and silicon dioxide.

6. The method of separating a phosphorylated peptide or a phosphorylated protein according to claim 1, wherein the metal oxide has a continuous porous structure.

7. The method of separating a phosphorylated peptide or a phosphorylated protein according to claim 1, wherein the metal oxide comprises an anatase crystal and/or an amorphous crystal and undergoes a weight reduction of 3 to 70 mg/g during a process of increasing the temperature by 40° C. per minute to 800° C. following heating at 130° C. for 15 minutes upon differential thermogravimetric analysis.

8. The method of separating a phosphorylated peptide or a phosphorylated protein according to claim 7, wherein the weight reduction is 4 to 20 mg/g.

9. A method of mass spectrometry of a phosphorylated peptide and/or a phosphorylated protein, comprising the steps of: supplying a sample containing a phosphorylated peptide and/or a phosphorylated protein separated by the method of separating a phosphorylated peptide or a phosphorylated protein according to any one of claims 1 to 8 to a mass spectrometer; and carrying out mass measurement of the separated phosphorylated peptide and/or the phosphorylated protein.

10. A method of separating a phosphorylated peptide or a phosphorylated protein, comprising the step of supplying a sample containing a phosphorylated peptide and/or a phosphorylated protein to a separation unit filled with titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis.

11. The method of separating a phosphorylated peptide or a phosphorylated protein according to claim 10, wherein the titanium oxide undergoes a weight reduction of 4 to 20 mg/g.

12. The method of separating a phosphorylated peptide or a phosphorylated protein according to claim 10, wherein the sample is supplied to the separation unit in the presence of an aliphatic hydroxycarboxylic acid.

13. The method of separating a phosphorylated peptide or a phosphorylated protein according to claim 10, wherein the titanium oxide has a continuous porous structure.

14. A chromatography stationary phase mainly consisting of titanium oxide comprising an anatase crystal and/or an amorphous crystal and undergoing a weight reduction of 3 to 70 mg/g during a process of increasing the temperature by 40° C. per minute to 800° C. following heating at 130° C. for 15 minutes upon differential thermogravimetric analysis.

15. The chromatography stationary phase according to claim 14, wherein the weight reduction is 4 to 20 mg/g.

16. The chromatography stationary phase according to claim 14, wherein the titanium oxide has a continuous porous structure.
